(19) United States
(12) Patent Application Publication
(10) Pub. No.: US 2015/0025353 A1
(43) Pub. Date: Jan. 22, 2015

(54) METHOD AND DEVICE FOR DETERMINING BODY COMPOSITION

(71) Applicants: Kenneth Alexander Johannes Salonius, Helsinki (FI); Riku Oskari Lehtomäki, Helsinki (FI)

(72) Inventors: Kenneth Alexander Johannes Salonius, Helsinki (FI); Riku Oskari Lehtomäki, Helsinki (FI)

(21) Appl. No.: 14/333,519

(22) Filed: Jul. 17, 2014

(30) Foreign Application Priority Data
Jul. 18, 2013 (FI) 20135780

(51) Int. Cl. A61B 5/053; A61B 5/00

(52) U.S. Cl. CPC A61B 5/0537; A61B 5/6804; A61B 5/681; A61B 5/742
USPC 600/388; 600/390; 600/547

(57) ABSTRACT

An arrangement (100) is for creating body composition images of a body part by placing the apparel (101) in direct contact with the desired body part. The results of the image are shown on separate mobile devices (102), such as e.g. a mobile phone, tablet computer or similar device. The image and derivative information can be calculated on the mobile device (102) or with external computation resources.

### TECHNICAL FIELD OF THE INVENTION

The invention relates to a method, device and system for determining body composition. Especially the invention relates to a method, device and system for determining internal body composition of different body compartments based on measurements on a skin using electrical impedance tomography imaging techniques.

### BACKGROUND OF THE INVENTION

Different kinds of solutions are known from prior art for providing information about the body composition, such as determining the development of muscle mass and fat mass during periods training. One of the known methods is to use a scale for weight measurement and/or waist circumference for abdominal fat measurement. Other physical measurements are body fat calipers and underwater weighting. Body fat calipers pinch the skin at several standardized points on the body to determine the thickness of the subcutaneous fat layer. These measurements are converted to an estimated body fat percentage by a set of equations. However, the accuracy of these estimates is more dependent on a person's unique body fat distribution than on the number of sites measured, and furthermore, the method is not able to measure visceral fat.

More advanced prior art scales include bio-impedance based body composition analysis (BIA), where current is fed and potential measurement on the surface are measured typically with 2-8 electrodes from feet and optionally palms to generate an estimate of the body composition. Other forms of bio-impedance devices for analysing abdominal fat include belt shaped measurement devices with cable connected instrumentation device for data collection. All of these give a number estimate of the measured body composition. However, BIA uses a statistical relationship between electrical properties of tissues and the target variable, it can be referred to as a prediction technique. Therefore, BIA equations are population-specific, and the accuracy of BIA results is considerably dependent on the agreement of physical characteristics, weight status, ethnicity and age between the subject and the reference population used to generate the BIA algorithm.

On the other hand bio-impedance based imaging, electrical impedance tomography, has been developed in the field of medical imaging mainly for medical diagnostic purposes. It has been applied to lung imaging, cardiac imaging and body composition analysis.

Electrical impedance tomography (EIT) is a non-invasive medical imaging technology that creates an image of conductivity distribution, permittivity distribution or combination of the two. Basic principle of electrical impedance tomography is that current is injected to a volume with one or more electrodes and voltage is measured on the electrodes placed on the surface of the volume. Creating impedance tomography image requires solving the inverse problem of volume conductance based on measured voltages from the boundary of the volume.

Prior art EIT body composition devices are based on EIT systems that typically have separate instrumentation part and electrodes that are connected with a cable to it. Wireless EIT instrumentation devices have been developed to overcome the problem of insulating the device from the mains power supplies.

There are however some disadvantages relating to the known prior art solutions. For example, traditional methods, scales, waist circumference, show results with plain numbers, which are not very illustrative. Also the body mass weight, waist circumference etc. do not contain information about the different tissue types and their changes in body.

More sophisticated bio-impedance based body composition analysis devices create estimates of the body composition based on measurements made on reference group and interpolating the results based on pre-saved database. Bio-impedance analyzers anyway do not solve the inverse problem to create an image, thus showing the results only in plain numbers and percentages.

Currently electrical impedance tomography is limited to hospital or ambulatory use only due to the need of having electrodes placed on the skin in known locations, and the electrodes are usually secured with adhesives to skin to ensure proper electrode-skin-connection, due to both operation needing trained personnel to use.

The current electrical impedance tomography devices moreover consist of electrode instance and separate measurement module that makes the size of the system unnecessary cumbersome for use outside hospitals. In addition many of the existing EIT devices consist also of separate electrodes or an electrode piece that is connected to a separate instrumentation device.

### SUMMARY OF THE INVENTION

An object of the invention is to alleviate and eliminate the problems relating to the known prior art. Especially the object of the invention is to provide a device for creating images of body composition and visualizing the results in a very illustrative way. In addition the object of the invention is to get in-depth information e.g. about the changes in the body in an easy way, such as determining the development of muscle mass and fat mass during periods training. The object is also to enable non-expert users to make measurements easily and advantageously without any adhesives or conductive gels, and minimize any misplacement of measuring electrodes due to human errors.

The object of the invention can be achieved by the features presented in this document and especially by the features of claims. The invention relates to a system for determining internal body composition of a patient according to claim 1, to a data acquisition device according to claim 8, to a method according to claim 10, and to a computer program product according to claim 11.

According to an embodiment for determining internal body composition of a patient a multichannel electrical impedance tomography device (data acquisition device) is used, which injects current through a body and measures the resulting voltages through a plurality of electrodes placed on the skin. The measured data is transferred to a data processing unit advantageously wirelessly to provide an image from said measurement raw data for representing the internal body composition of the patient. The data processing unit advantageously sends the image to a display device for displaying it.

The data acquisition device comprises advantageously at least one pair of electrodes configured to inject electric current into a volume of the body of the patient, and at least one pair of electrodes configured to measure the resulting voltage on the skin of said patient. According to an advantageous embodiment the data acquisition device is implemented by a multilayer garment supporting the electrodes, where the multilayer garment comprises at least one stretchable layer and one non-stretchable corrugated layer. The layers are coupled with each other in numerous portions so that the corrugation portions of the non-stretchable corrugated layer between the coupling portions are free from the stretchable layer. The electrodes are advantageously arranged into the non-stretchable layers at the coupling portions. According to an embodiment the data acquisition device is e.g. a belt like device or other garment like device, such as shirt, harness, vest, strap or the like.

According to an embodiment the data processing unit receiving the measurement data from the acquisition device and also providing the image is a mobile communication device, such as mobile phone, tablet computer or the like. According to another embodiment the data processing unit may be an external data processing unit, such as a cloud system or other server system having high data processing power, whereupon the data acquisition device sends the measurement data to the external data processing unit for processing and image formation. It is to be noted that the data processing unit may advantageously send the data to the external data processing unit via a mobile communication device, such as mobile phone, using mobile telecommunication network. The generated image is sent to the display device, which is according to an advantageous embodiment the display of the mobile communication device.

The image is reconstructed based on the measurements, but possibly also on any other additional data such as age, weight, height, sex, waist circumference etc. According to an embodiment the data processing unit may also reconstruct the shape of the body based on the measurements and thereby provide an image or even series of images.

The data acquisition device comprises a plurality of electrodes, where the same electrodes can be used both for injecting current and measuring the resulting voltages e.g. subsequently. The data acquisition device may comprise e.g. six to hundreds of electrodes, advantageously at least 8 and more advantageously at least 16 electrodes. The more electrodes the more detailed image can be provided. Advantageously multiple periods of the electrode voltages are measured to ensure long enough sample of the signal. In addition a reference voltage from the current injection is also determined in order to measure a phase delay of the electrode voltages. The electrodes used for injecting the current and measuring the voltage are advantageously varied in multiple periods measurement so that at first a first set of electrodes is used for injecting and a other set of electrodes (e.g. rest of the electrodes) for measuring, and at second another set of electrodes is used for injecting and again other set of electrodes (e.g. rest of the electrodes) for measuring. It is to be noted that multiple variation for injecting and measuring electrodes is possible e.g. for measuring different parameters for the image to be provided from the measured data.

In addition the data acquisition device comprises a current source configured to supply current with constant frequency in a range of 10-200 kHz, more advantageously in a range of 50-100 kHz. The frequency range is noticed to be specially advantage for measurement, because the current with these frequencies penetrates easily into the body and in addition the measurement with these frequencies is easy and reliable to determine even without using any adhesives or conductive gels.

According to an embodiment the non-stretchable layer of the data acquisition device comprises conductive paths for supplying electric current to the electrodes configured to inject electric current into the volume of the body of the patient, as well as transferring measured voltage from at least one pair of electrodes configured to measure said resulting voltage on the skin of said patient. The conductive paths comprise advantageously metallic or other electrically conductive material, such as copper or silver particles.

According to an embodiment the data acquisition device typically includes battery or other form of integrated power supply, as well as data communication means to ensure wireless operation. The data processing unit is configured to generate the image by solving the conductivity inverse problem.

It is to be understood that an external processing and storage capacity (e.g. cloud computing system) may be used by the data processing unit, especially by the mobile device while generating the image from the measurement data. The mobile device may be for example a Laptop, Tablet, Smartphone or other similar consumer electronics.

In addition it is to be noted that the measured voltages form a dataset which is an approximation of the voltage distribution on the skin, but with knowing (measuring or knowing beforehand) the currents injected on the boundary and the measured voltage distribution, the internal conductivity distribution can be found by finding a solution to the inverse conductivity problem, i.e. solving the Calderon's problem. However the reconstruction of the voltage distribution data into a tomographic image is computationally intensive operation. However, this can be overcome by the current invention with the aid of the data processing unit being external from the data acquisition device, such as with the aid of an external mobile device. Additionally this mobile device may benefit from external computation resources (e.g. cloud computing) to create the image from the measurement data. The display of the mobile device can, however, be used to show the visualization as MRI-like cross-sectional image of the body.

The present invention offers advantages over the known prior art. By using electrical impedance tomography in body composition analysis actual images of the desired body part can be created. In addition by incorporating the EIT instrumentation device to the electrode attachment piece the size of the EIT device can be reduced remarkably. Using mobile device as user interface and processing unit the size and cost of the EIT unit can further be reduced. In addition it is to be noted that the more secure the electrode skin contact is the better results the device will give.

Especially the embodiments of the invention can be easily and in a low-cost way to use for visualizing body composition. It enables users to track and follow visually developments of composition of different body compartments without the need of expensive equipment or trained professionals using the device. Such changes include changes in the amount of adipose tissue in abdomen when person starts to exercise. Other possible rapid changes in internal body composition might include such as internal hemorrhage or excess amounts of other body fluids in body cavities. Muscle sizes, muscle abnormalities and other soft tissue abnormalities can also be determined. The data acquisition device according to the invention may be used e.g. for personal use at home or at gym for example, but also in military and rescue areas or the like, where possible changes in the body of the user, such as internal haemorrhage can be determined and thereby an alarm or other notice may be provided to the user or operational centre in order to warning if there appears abnormal or too rapid changes in the body of the user.

In addition the stretchable nature and structure of the data acquisition device ensures high quality contact between the electrodes and the body without requiring any adhesives or conductive gels, which is great advantage over the known techniques and highly preferable feature outside hospital and ambulatory environments.

Furthermore the stretchable data acquisition device as an electrode carrier according to the invention ensures easily correct placements of the electrodes. The stretchable data acquisition device also allows the measurements without causing stress to the conductive paths connecting the electrodes and a terminal or other electronic or controlling unit of the data acquisition device, such as a low-power MUC with sufficient processing capabilities for filtering purposes, for example.

The data acquisition device may be e.g. belt integrated to allow imaging of the subject over on cross-sectional plane. Other configurations may include e.g. harness-type carriers that allow placing the electrodes over multiple planes to allow volumetric imaging modes.

The exemplary embodiments of the invention presented in this document are not to be interpreted to pose limitations to the applicability of the appended claims. The verb “to comprise” is used in this document as an open limitation that does not exclude the existence of also unrecited features. The features recited in the document are mutually freely combinable unless otherwise explicitly stated.

### BRIEF DESCRIPTION OF THE DRAWINGS

Next the invention will be described in greater detail with reference to exemplary embodiments in accordance with the accompanying drawings, in which

FIG. 1 illustrates a block diagram of an exemplary system for providing internal body composition determination according to an advantageous embodiment of the invention,

FIG. 2 illustrates a block diagram of another exemplary system for providing internal body composition determination according to an advantageous embodiment of the invention,

FIG. 3 illustrates an exemplary data acquisition device for determining internal body composition according to an advantageous embodiment of the invention,

FIG. 4 illustrates an exemplary measuring arrangement for providing internal body composition determination according to an advantageous embodiment of the invention, and

FIG. 5 illustrates an exemplary image reconstructed from measured data according to an advantageous embodiment of the invention.

### DETAILED DESCRIPTION

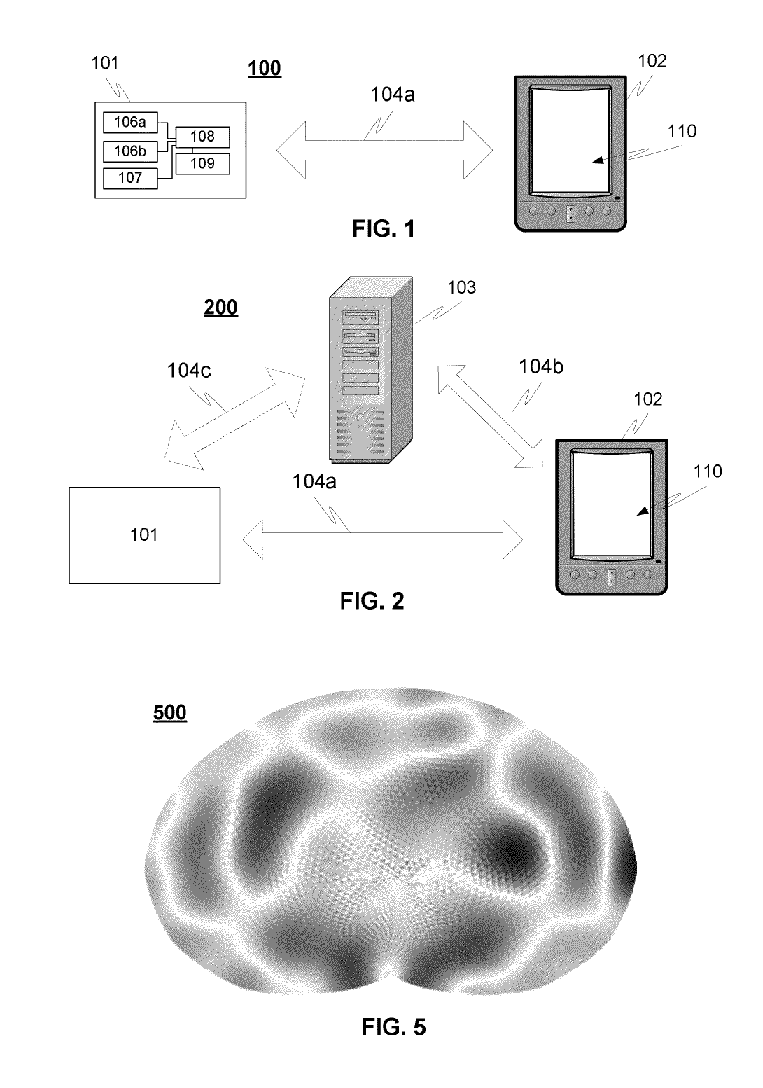

FIG. 1 illustrates a block diagram of an exemplary system 100 and method for providing internal body composition determination according to an advantageous embodiment of the invention, where the data acquisition device 101, such as an EIT integrated apparel, comprises a plurality of electrodes 106a (for current injecting), 106b (for voltage measuring), a power and current source 107, such as a battery, a controlling unit 108 controlling the current injection as well as measurements, and data communication means 109, advantageously wireless data communication means using for example Bluetooth technique or the like. According to an example the data acquisition device is configured to sample the electrodes with solid state switches connecting the electrodes that are measuring and injecting current to respective modules.

In addition in the system 100 and method the measured data is transferred 104a104c from the data acquisition device 101 via the data communication means 109 to the data processing unit 102, 103, which is configured to provide an image from the measured and transferred raw data representing the internal body composition of the patient. The data processing unit 102, 103 is also configured to send the produced image to a display device 110 for displaying it. The display device 110 may be a separate external display device or for example display device of the data processing unit, such as a display device 110 of a mobile phone or tablet computer 102.

It is to be noted that according to an embodiment said data processing unit 102 is a mobile communication device, which has enough data processing power to produce the image, as is illustrated in FIG. 1, but according to another embodiment said data processing unit 103 is an external data processing unit having great data processing power, such as cloud or server system, as illustrated in a system 200 of FIG. 2, whereupon the mobile communication device 102 is advantageously used for data transferring between the data acquisition device 101 and the data processing unit 103 and possibly also for displaying the image via its display device 110. Again it is to be noted that the data acquisition device 101 may be configured to send 104c the measured data directly to the data processing unit 103 for image production, which again may be configured to send 104b the produced image to the mobile communication device 102 or the like for displaying it via the displaying device 110.

According to an advantageous method the measured raw data is first transmitted 104a to a mobile device 102, as is illustrated in FIG. 2, where the mobile device 102 again communicates 104b at least part of it to the third party, such as cloud system 103 for processing, which again returns 104b a processed form (such as a graphical tomographic image) back to the mobile device 102 for displaying it to the user via its graphical interface 110. The advantage is that the data acquiring device 101 does need to comprise any communication means configured to enable communication channel with the external server 103.

In addition the data processing unit 102, 103 advantageously comprises suitable computer program product configured to process the measured data in order to provide the image, when said computer program is run on the data processing unit. The processing advantageously comprises an iterative method for solving partial differential equations numerically comprising the following steps:
building a body model that divides the respective space to an array of arbitrary polytopes,
placing a computational abstraction of the measurement device to the body model,
including prior information of the subject to help solving the numerical model, and
solving the model with the parametrization that comprises the body model, measurement device model, prior data and internal admittivity distribution.

Additionally the parametrization may include explicit formulation of the boundary shape to recover the boundary shape of the model. After the above steps a voxel grid may be reconstructed that allows transforming the arbitrary polytope data to normal computer images or volumetric models.

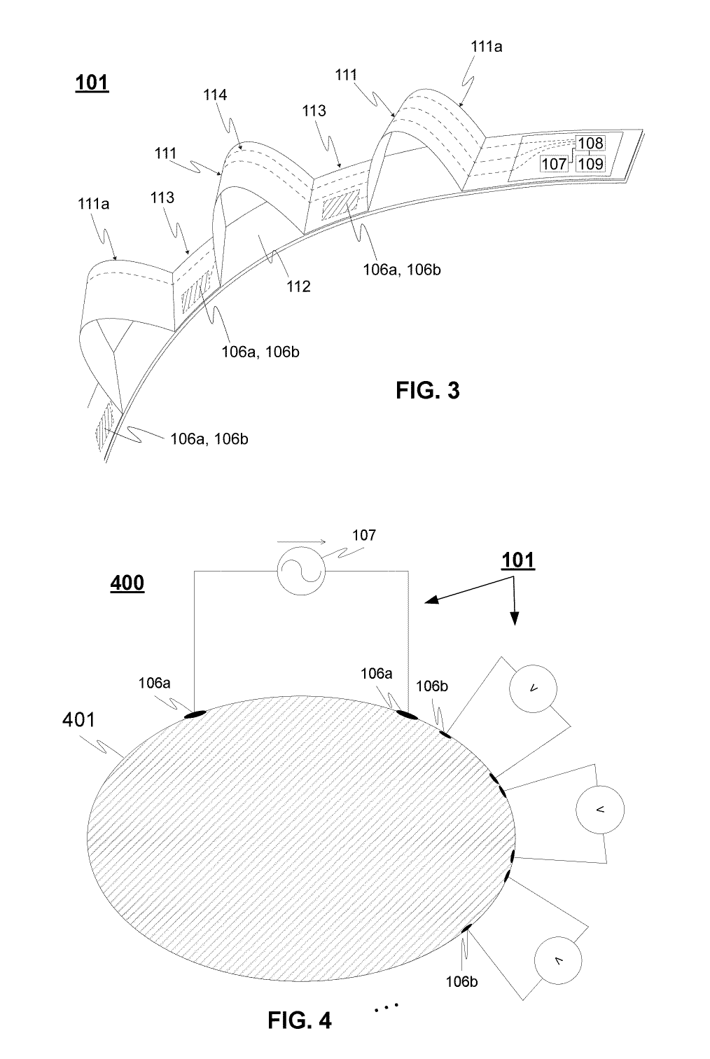

FIG. 3 illustrates an exemplary data acquisition device 101 for gathering data for internal body composition determination according to an advantageous embodiment of the invention. The data acquisition device 101 advantageously comprises a multilayer 111, 112 garment configured for supporting the electrodes 106a, 106b. The multilayer garment comprises at least one stretchable layer 112 and one non-stretchable corrugated layer 111. The layers are coupled with each other in numerous portions 113 so that the corrugation portions 111a of said non-stretchable corrugated layer 111 between the coupling portions 113 are free from the stretchable layer 112. The electrodes 106a, 106b are advantageously arranged into the non-stretchable layers at the coupling portions 113, whereupon possibly induced stress is minimized.

The non-stretchable layer 111 advantageously comprises conductive paths 114 for supplying electric current to the electrodes 106a, 106b configured to inject electric current into the volume of the body of the patient, as well as transferring measured voltage from at least one pair of electrodes 106a, 106b configured to measure said resulting voltage on the skin of said patient.

The data acquisition device may be integrated for example to a garment wearable by the user, such as a belt, harness, shirt, bra or wristband.

FIG. 4 illustrates an exemplary measuring arrangement 400 for providing internal body composition determination according to an advantageous embodiment of the invention, where the data acquisition device 101 is set around the body 401 to be measured. As can be seen the current source 107 injects current to the injecting electrodes 106a, whereas the other electrodes 106b are used for voltage measuring.

FIG. 5 illustrates an example of an internal body composition as a reconstructed image 500 from measured data according to an advantageous embodiment of the invention, where the shape and different portions of the body (composition of the body) can be seen via different grey values.

The invention has been explained above with reference to the aforementioned embodiments, and several advantages of the invention have been demonstrated. It is clear that the invention is not only restricted to these embodiments, but comprises all possible embodiments within the concept and scope of the inventive thought and the following patent claims. Especially it is to be noticed that there are different forms and shapes that the device can be made into.

1. A system for determining internal body composition of a patient, the system comprising:
a data acquisition device configured to gather measurement signals as raw data on the skin of the patient,
a data processing unit for receiving said raw data from the data acquisition device, where said data processing unit is configured to provide an image from said raw data representing the internal body composition of the patient and to send it to a display device for displaying it, and
wherein
said data acquisition device comprises
at least one pair of electrodes configured to inject electric current into a volume of the body of the patient,
at least one pair of electrodes configured to measure the resulting voltage on the skin of said patient,
multilayer garment supporting said electrodes, where said multilayer garment comprises at least one stretchable layer and one non-stretchable corrugated layer, said layers being coupled with each other in numerous portions so that the corrugation portions of said non-stretchable corrugated layer between the coupling portions are free from said stretchable layer and wherein said electrodes are arranged into the non-stretchable layers at the coupling portions.

2. A system of claim 1, wherein said data processing unit is a mobile communication device.

3. A system of claim 1, wherein said data processing unit is an external data processing unit, like a cloud system, and wherein said data acquisition device is configured to send the raw data to said data processing unit via a mobile communication device.

4. A system of claim 1, wherein said data processing unit is configured to provide said image to the display of said mobile communication device.

5. A system of claim 1, wherein said non-stretchable layer comprises conductive paths for supplying electric current to the electrodes configured to inject electric current into the volume of the body of the patient, as well as transferring measured voltage from at least one pair of electrodes configured to measure said resulting voltage on the skin of said patient.

6. A system of claim 1, wherein said data acquisition device comprises a current source configured to supply current with constant frequency in a range of 10-200 kHz, more advantageously in a range of 50-100 kHz.

7. A system of claim 1, wherein said system is configured to measure multiple periods of the electrode voltages and a reference voltage from the current injection in order to measure a phase delay of the electrode voltages.

8. A data acquisition device for determining internal body composition of a patient, said data acquisition device being configured to gather measurement signals as raw data on the skin of the patient, wherein said data acquisition device comprising:
at least one pair of electrodes configured to inject electric current into a volume of the body of the patient,
at least one pair of electrodes configured to measure the resulting voltage on the skin of said patient, and
multilayer garment supporting said electrodes, where said multilayer garment comprises at least one stretchable layer and one non-stretchable corrugated layer, said layers being coupled with each other in numerous portions so that the corrugation portions of said non-stretchable corrugated layer between the coupling portions are free from said stretchable layer and wherein said electrodes are arranged into the non-stretchable layers at the coupling portions.

9. A data acquisition device of claim 8, wherein said data acquisition device comprises a garment wearable by said patient for the determination, such as a belt, harness, shirt, bra or wristband.

10. Method for determining internal body composition of a patient, the method comprising:
gathering measurement signals as raw data on the skin of the patient by a data acquisition device comprising a plurality of electrodes, and
receiving said raw data from the data acquisition device by a data processing unit for providing an image from said raw data representing the internal body composition of the patient and sending said image to a display device for displaying it,
wherein
electric current is injected into a volume of the body of the patient by at least one pair of electrodes,
the resulting voltage is measured on the skin of said patient by at least one pair of electrodes, and
said electrodes are supported by a multilayer garment, where said multilayer garment comprises at least one stretchable layer and one non-stretchable corrugated layer, said layers being coupled with each other in numerous portions so that the corrugation portions of said non-stretchable corrugated layer between the coupling portions are free from said stretchable layer and wherein said electrodes are arranged into the non-stretchable layers at the coupling portions.

11. Computer program product for determining internal body composition of a patient, the computer program product comprising computer readable code means configured to:
receive raw data from a data acquisition device for providing an image from said raw data representing the internal body composition of the patient and send said image to a display device for displaying it, where said data acquisition device is configured to gather measurement signals as raw data on the skin of the patient by using at least one pair of electrodes for injecting electric current into a volume of the body of the patient and at least one pair of electrodes to measure the resulting voltage on the skin of said patient,
whereupon the computer readable code means is additionally configured to
solving the shape of the measured body,
solving the electrode locations and contact impedances,
solving the internal admittivity distribution of the injected current in the body, and
reconstructing said image using these results,
when said computer code means is run on a data processing unit.
